(19) United States
(12) Patent Application Publication
(10) Pub. No.: US 2023/0059539 A1
(43) Pub. Date: Feb. 23, 2023

(54) RESPIRATORY DRUG DELIVERY DEVICE AND METHOD FOR AUTOMATING DRUG DELIVERY

(71) Applicant: National Tsing Hua University, Hsinchu (TW)

(72) Inventors: Chi-Shuo CHEN, Hsinchu (TW); Hui-Ling LIN, Hsinchu (TW); Geng-Yue LI, Hsinchu (TW); Megha JHUNJHUNWALA, Hsinchu (TW); Rong-Shing CHANG, Hsinchu (TW); Chaang-Ray CHEN, Hsinchu (TW); Sheng-Ting HUNG, Hsinchu (TW)

(21) Appl. No.: 17/714,099

(22) Filed: Apr. 5, 2022

(30) Foreign Application Priority Data
Aug. 12, 2021 (TW) 110129698

(51) Int. Cl. A61M 16/06; A61M 16/08; A61M 15/00

(52) U.S. Cl. CPC A61M 16/0666; A61M 16/0875; A61M 15/001; A61M 2016/003

(57) ABSTRACT

A respiratory drug delivery device and a method for automating drug delivery are provided. The respiratory drug delivery device may be utilized for delivering a drug to a patient's respiratory tract. The respiratory drug delivery device comprises an inhalation element, an air pipe, a sound sensor, and a drug delivery module, the drug delivery module delivers an aerosolized medicine when the patient inhales based on a sound signal.

### CROSS REFERENCE TO RELATED APPLICATION

This application claims the benefit of Taiwan Patent Application Serial No. 110129698 filed on Aug. 12, 2021, the subject matter of which is incorporated herein by reference.

### BACKGROUND OF THE INVENTION

### 1. Field of the Invention

The present invention relates to a respiratory drug delivery device and a method for automating drug delivery to a respiratory system; particularly, to a respiratory drug delivery device for intermittent administration and a method for automating drug delivery to a respiratory system using the respiratory drug delivery device.

### 2. Description of Related Art

The aerosol therapy device used for respiratory therapy in current clinical practice usually supplies aerosol drugs continuously. The aerosol drug is continuously provided even when the patient is exhaling, therefore, the drug delivery rate is not efficient. On average, only 20% to 30% of the aerosol drug enters into the patient's body through the respiratory system. The residual drug cannot be used, thus causing a serious problem of waste of drugs.

Accordingly, a novel drug delivery device is needed for accurately administering the aerosol drug at the appropriate breathing period based on different ages or different disease conditions of patients to improve the delivery efficiency.

### SUMMARY OF THE INVENTION

The present invention provides a respiratory drug delivery device for delivering a drug to a patient's respiratory tract, comprising: an inhalation part; an air pipe, being connected to the inhalation part; a sound sensor, being disposed in the air pipe for detecting a wind shear from the patient's breath and generating a sound signal; and a drug delivery module, being connected to the air pipe and the sound sensor, wherein the drug delivery module nebulize the drug into a nebulized drug and output the nebulized drug to the air pipe; wherein the drug delivery module output the nebulized drug at least when the patient inhales according to the sound signal.

In one embodiment, the sound signal includes a plurality of breathing cycles, each of the breathing cycles sequentially includes an inhalation section, a pause section, and an exhalation section; wherein the drug delivery module outputs the nebulized drug at least at the inhalation section.

In one embodiment, the drug delivery module outputs the nebulized drug at 0.1 to 5 seconds before the beginning of the inhalation section until the end of the inhalation section.

In one embodiment, the drug delivery module outputs the nebulized drug at 0.1 to 5 seconds before the beginning of the inhalation section until 0.5 to 3 seconds before the end of the inhalation section.

In one embodiment, the nebulized drug is at least one selected from a group consisting of a nebulized micronized drug, a nebulized water-soluble drug, and a nebulized drug suspension.

In one embodiment, the drug delivery module includes a nebulizer for generating the nebulized drug.

In one embodiment, a nozzle diameter of the nebulizer is below 10 μm.

In one embodiment, the nebulizer is an ultrasonic vibrating atomizer.

In one embodiment, the inhalation part is a nasal mask, an oronasal mask, or a nasal catheter.

The respiratory system provided by the present invention may be connected to the conventional respirator and delivering the nebulized drug to the patient's respiratory tract when assisting the patient's breathe.

The present invention further provides a method for automating drug delivery to the respiratory system, comprising: step (1): providing a respiratory drug delivery device, including an inhalation part; an air pipe being connected to the inhalation part; a sound sensor being disposed in the air pipe; and a drug delivery module outputting a nebulized drug to the air pipe; step (2): collecting a respiratory flow with the air pipe through the inhalation part, detecting a wind shear from the patient's breath and generating a sound signal using the sound sensor; and step (3): determining a breathing cycle of the patient from the sound signal; outputting the nebulized drug to the air pipe at least when the patient inhales; and outputting the nebulized to the patient's respiratory system via the inhalation part.

In one embodiment, in step (2), a respiratory frequency of the patient is 0.2 to 1 Hz.

In one embodiment, in step (2), the respiratory flow is generated from the patient's nose and delivered to the air pipe.

In one embodiment, in step (3), the sound signal includes a plurality of breathing cycles, each of the breathing cycles sequentially includes an inhalation section, a pause section, and an exhalation section, wherein the drug delivery module outputs the nebulized drug at least at the inhalation section.

In one embodiment, in step (3), the drug delivery module outputs the nebulized drug at 0.1 to 5 seconds before the beginning of the inhalation section until the end of the inhalation section.

In one embodiment, in step (3), the drug delivery module outputs the nebulized drug at 0.1 to 5 seconds before the beginning of the inhalation section until 0.5 to 3 seconds before the end of the inhalation section.

### BRIEF DESCRIPTION OF THE DRAWINGS

FIG. 1 is a schematic diagram of the respiratory drug delivery device of one embodiment of the present invention;

FIG. 2 is a schematic diagram of the respiratory drug delivery device of another embodiment of the present invention;

FIG. 3 is a signal diagram detected by the respiratory drug delivery device of one embodiment of the present invention; and

FIG. 4 is a schematic diagram of the breathing cycle of the sound signal detected by the respiratory drug delivery device of one embodiment of the present invention.

### DETAILED DESCRIPTION OF THE PREFERRED EMBODIMENT

Hereafter, examples will be provided to illustrate the embodiments of the present invention. The advantages and effects of the invention will become more apparent from the disclosure of the present invention. Other various aspects also may be practiced or applied in the invention, and various modifications and variations can be made without departing from the spirit of the invention based on various concepts and applications.

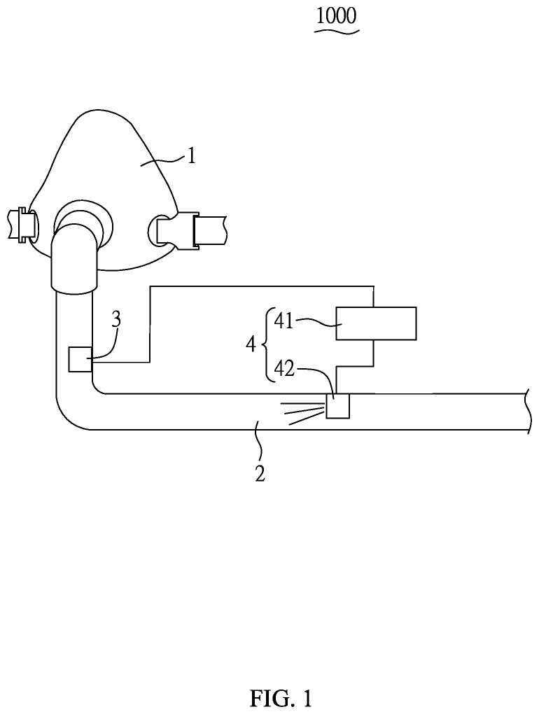

The respiratory drug delivery device 1000 provided by one embodiment of the present invention is illustrated in FIG. 1, wherein the respiratory drug delivery device 1000 comprises an inhalation part 1, an air pipe 2, a sound sensor 3, and a drug delivery module 4.

In the present embodiment, the inhalation part 1 is an oronasal mask covering the patient's mouth and nose. However, in other embodiments, the inhalation part 1 may be a nasal mask, or a nasal catheter, which can be chosen according to the patient's conditions as long as the inhalation part 1 is interconnected to the patient's respiratory system and can collect the patient's breath.

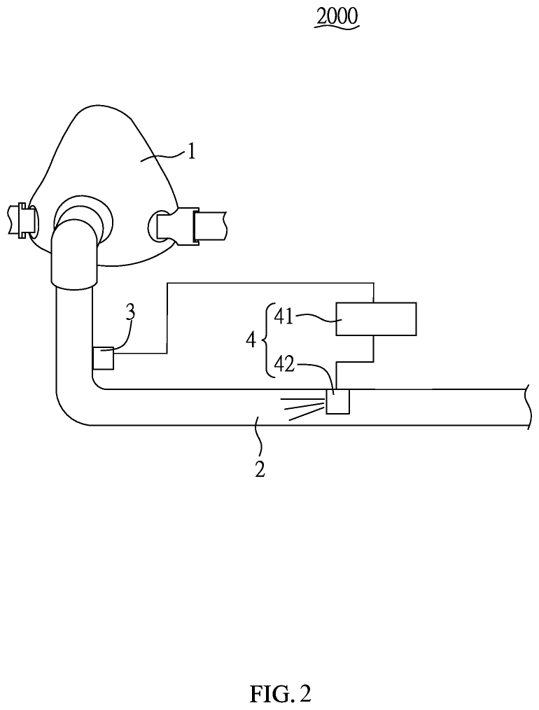

The air pipe 2 is connected to the inhalation part 1, the sound sensor 3 is a microphone being disposed on the inner surface of the air pipe 2. The sound sensor 3 is preferably disposed adjacent to the inhalation part 1 to detect the wind shear generated by the patient's breathes and transform the detected wind shear into a sound signal and send it to the drug delivery module 4. The wind shear sound of the patient's breathes was generated when the airflow generated by the inhalation and exhalation passes through the sound sensor 3. In other embodiments, the sound sensor 3 of the respiratory drug delivery device 2000 illustrated in FIG. 2 is disposed on the outer surface of the air pipe 2 for detecting the wind shear sound of the patient's breathes.

The drug delivery module 4 includes a signal processor 41 and a nebulizer 42. The signal processor 41 receives the sound signal collected by the sound sensor 3 and then processing the sound signals. The sound signal is then divided into a plurality of breathing cycles, and each of the breathing cycles sequentially includes an inhalation section, a pause section, and an exhalation section. The nebulizer 42 is disposed in the air pipe 2, and is preferably disposed away from the inhalation part 1 and the sound sensor 3. A nebulized drug is outputted by the nebulizer 42 at the inhalation section and is delivered to the inhalation part 1 through the air pipe 2 and further to the patient's respiratory system. However, in the exhalation section, the nebulizer 42 stops outputting the nebulized drug to improve the efficiency of drug delivery and avoid wasting. Particularly, the nebulizer is an ultrasonic vibrating nebulizer for nebulizing a drug, wherein the drug can be a micronized drug, a water-soluble drug, or a drug suspension, and a nozzle diameter of the nebulizer is below 10 μm.

The respiratory drug delivery device of the present embodiment can be further connected to a conventional respirator and delivering the nebulized drug to the patient's respiratory system when assisting the patient's breathe. However, the present invention is not limited thereto and can be configured as needed.

In another embodiment, the nebulized drug may be output by the nebulizer 42 at 0.1 to 5 seconds before the beginning of the inhalation section until the end of the inhalation section. That is, the nebulized drug may be output at 0.1 to 5 seconds before the end of the exhalation section until the end of the inhalation section. Accordingly, the drug delivery efficiency may be further improved because the nebulized drug is filled in the inhalation part 1 (the oronasal mask) when the patient starts to inhale.

In yet another embodiment, the nebulized drug may be output by the nebulizer 42 at 0.1 to 5 seconds before the beginning of the inhalation section until 0.5 to 3 seconds before the end of the inhalation section. Accordingly, the drug delivery efficiency may be further improved by avoiding the waste of the drug because the nebulized drug is filled in the inhalation part 1 when the patient starts to inhale and prevent the excessive residual nebulized drug from filling the inhalation part 1 at the beginning of the exhalation section.

The method for automating drug delivery to a respiratory system using the respiratory drug delivery device 1000 of the present invention is described in the following paragraphs, wherein the method comprises the steps of:

Step (1): providing a respiratory drug delivery device, including an inhalation part, an air pipe being connected to the inhalation part, a sound sensor being disposed in the air pipe, and a drug delivery module outputting a nebulized drug to the air pipe. The respiratory drug delivery device and the components thereof are described in the previous paragraphs.

Step (2): collecting a respiratory flow using the air pipe through the inhalation part, detecting a wind shear from the patient's breath, and generating a sound signal using the sound sensor. In the present embodiment, the respiratory flow is emitted from the patient's nose and flows into the air pipe. The sound sensor is disposed on the inner surface or the outer surface of the air pipe to avoid detecting noises generated from the patient's heartbeat or other medical appliances when detecting the wind shear of the respiratory flow. Accordingly, it can be assured that the inhalation section, the pause section, and the exhalation section of the sound signal generated from the sound sensor are accurate. Furthermore, the respiratory frequency of the patient usually is 0.2 to 1 Hz, that is, if the detected respiratory frequency exceeds this range, it will be necessary to check whether the sound sensor detects noises other than the breathing wind shear or whether the device is malfunctioning.

Step (3): determining a breathing cycle of the patient from the sound signal; outputting the nebulized drug to the air pipe at least when the patient inhales for delivering the nebulized to the patient's respiratory system via the inhalation part. Particularly, the drug delivery module outputs the nebulized drug to the patient's respiratory system through the inhalation part at the inhalation section and stops outputting the nebulized drug at the exhalation section for improving the drug delivery efficiency and avoiding drug wasting. Similarly, in another embodiment, the drug delivery module outputs the nebulized drug at 0.1 to 5 seconds before the beginning of the inhalation section until the end of the inhalation section. In yet another embodiment, the drug delivery module outputs the nebulized drug at 0.1 to 5 seconds before the beginning of the inhalation section until 0.5 to 3 seconds before the end of the inhalation section. Accordingly, the drug delivery efficiency may be further improved to reduce the waste of the drug.

[Evaluation of the Drug Delivery Efficiency]

The present embodiment utilized the breathing simulator to simulates the patient's breathing The respiratory drug delivery device of the present invention was connected to the breathing simulator via the inhalation part. The breathing simulator was used to simulate the breathing of a healthy patient and had a tube connected to the respiratory drug delivery device. The tube was provided with a filter paper disposed inside for absorbing the nebulized drug, wherein the weight of the drug absorbed by the filter paper was measured for evaluating the drug delivery efficiency. The settings of the breathing simulator were as follows: the inhalation/exhalation ratio (I/E ratio) was 1/2.5; the volume of the inhaled or exhaled air in each breathing cycle was 410 mL; the total amount of water, used for simulating the drug, was 1 mL. The water used for simulating the drug became nebulized water after being nebulized.

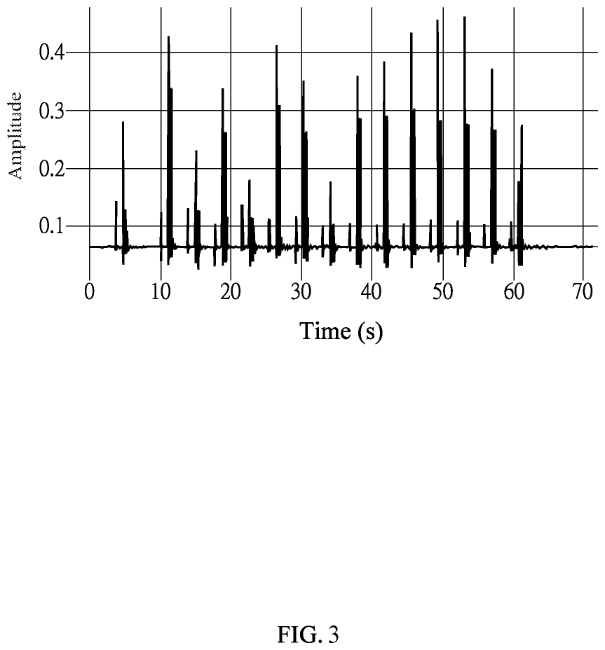

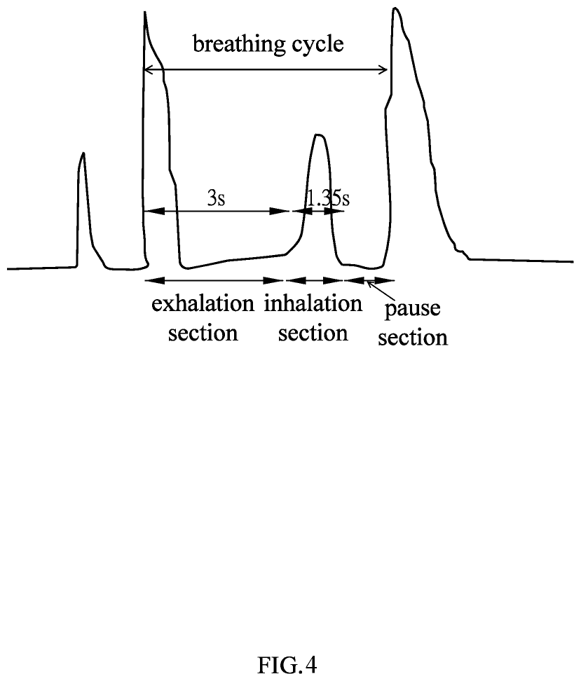

When the airflow generated by the breathing simulator entered the air pipe, the sound signal generated by the sound sensor after detecting the wind shear sound was shown in FIG. 3, and one of the breathing cycles of the sound signal was shown in FIG. 4. As shown in the figures, the inhalation section lasted for 1.35 seconds, the exhalation section lasted for 3 seconds, and a short pause section was between the inhalation section and the exhalation section. The present evaluation was repeated six times which included Example 1 to Example 6. From Example 1 to Example 6, the drug delivery module outputted the nebulized water into the air pipe and entered the tube of the breathing simulator within 1.35 seconds of the inhalation section. The nebulized water was absorbed by the filter paper along with the airflow of stimulated inhalation. The weight of the filter paper before outputting the nebulized water and after the nebulized water ran out were measured for calculation of the drug delivery efficiency. The calculation formula for the drug delivery efficiency was:

Drug delivery efficiency (%)=(weight of the filter paper after drug delivery−weight of the filter paper before drug delivery)/weight of water.

In addition, the present evaluation included eight comparative examples (Comparative example 1 to Comparative example 8) which are tested with continuous drug delivery. Similarly, the weight of the filter paper before outputting the nebulized water and after the nebulized water ran out were measured for calculation of the drug delivery efficiency. The drug delivery efficiency of the Examples and the Comparative examples were recorded in Table 1.

TABLE 1

| | Weight of the filter | Weight of the filter | Drug |
|---|---|---|---|
| | paper before outputting | paper after outputting | delivery |
| | the nebulized water | the nebulized water | efficiency |
| | (g) | (g) | (%) |
| Example 1 | 23.1206 | 23.7131 | 59.25 |
| Example 2 | 23.1380 | 23.7072 | 56.92 |
| Example 3 | 23.0632 | 23.6448 | 58.16 |
| Example 4 | 23.1299 | 23.7153 | 58.54 |
| Example 5 | 22.9990 | 23.6576 | 65.86 |
| Example 6 | 23.0860 | 23.7805 | 69.45 |
| Comparative | 23.0456 | 23.2675 | 22.22 |
| example 1 | | | |
| Comparative | 22.9708 | 23.1987 | 22.79 |
| example 2 | | | |
| Comparative | 23.1120 | 23.3456 | 23.36 |
| example 3 | | | |
| Comparative | 23.0440 | 23.2778 | 23.38 |
| example 4 | | | |
| Comparative | 23.0890 | 23.3109 | 22.19 |
| example 5 | | | |
| Comparative | 23.0818 | 23.3523 | 27.05 |
| example 6 | | | |
| Comparative | 23.1428 | 23.3941 | 25.13 |
| example 7 | | | |
| Comparative | 23.0760 | 23.3020 | 22.6 |
| example 8 | | | |

According to the evaluation results shown in Table 1, the drug delivery efficiencies of Examples 1 to 6, which only outputted the nebulized drug at the inhalation section, were significantly greater than that of Comparative examples 1 to 8. It is proven that the respiratory drug delivery device provided by the present invention is capable of increasing the drug delivery efficiency.

In the next evaluation, the settings of the breathing simulator were as follows: the inhalation/exhalation ratio (I/E ratio) was 1/2.5; the volume of the inhaled or exhaled air in each breathing cycle was 250 mL, 500 mL, and 800 mL; the total amount of water, used for simulating the drug, was 1 mL. The water used for simulating the drug became nebulized water after being nebulized. Similarly, the drug delivery module outputted the nebulized water into the air pipe and entered the tube of the breathing simulator within 1.35 seconds of the inhalation section. The nebulized water was absorbed by the filter paper along with the airflow of stimulated inhalation. The weight of the filter paper before outputting the nebulized water and after the nebulized water ran out were measured for calculation of the drug delivery efficiency. In the present evaluation, the drug delivery module only outputted the nebulized water into the air pipe and entered the tube of the breathing simulator within 1.35 seconds of the inhalation section in Examples, however, Comparative examples were tested with continuous drug delivery. Three Examples and three Comparative examples, which were Example 7 to Example 9 and Comparative example 9 to Comparative example 11, were tested with 250 mL volume of the inhaled or exhaled air; three Examples and three Comparative examples, which were Example 10 to Example 12 and Comparative example 12 to Comparative example 14, were tested with 500 mL volume of the inhaled or exhaled air; and three Examples and three Comparative examples, which were Example 13 to Example 15 and Comparative example 15 to Comparative example 18, were tested with 800 mL volume of the inhaled or exhaled air. The results were shown in Table 2.

TABLE 2

| | | Weight of the filter | Weight of the filter | |
|---|---|---|---|---|
| | | paper before | paper after | Drug |
| | Volume of | outputting the | outputting the | delivery |
| | the air | nebulized water | nebulized water | efficiency |
| | (mL) | (g) | (g) | (%) |
| Example 7 | 250 | 23.0025 | 23.7577 | 75.52 |
| Example 8 | 250 | 23.0599 | 23.8242 | 76.43 |
| Example 9 | 250 | 23.0692 | 23.8180 | 74.88 |
| Comparative | | | | |
| example 9 | 250 | 23.0219 | 23.4618 | 43.99 |
| Comparative | | | | |
| example 10 | 250 | 23.1196 | 23.5450 | 42.54 |
| Comparative | 250 | 23.1572 | 23.5666 | 40.94 |
| example 11 | | | | |
| Example 10 | 500 | 24.9836 | 25.6452 | 66.16 |
| Example 11 | 500 | 25.0870 | 25.7890 | 70.2 |
| Example 12 | 500 | 24.9096 | 25.6285 | 71.89 |
| Comparative | 500 | 25.0906 | 25.4643 | 37.37 |
| example 12 | | | | |
| Comparative | 500 | 24.9232 | 25.3083 | 38.51 |
| example 13 | | | | |
| Comparative | 500 | 24.7765 | 25.1677 | 39.12 |
| example 14 | | | | |
| Example 13 | 800 | 23.0863 | 23.6005 | 51.42 |
| Example 14 | 800 | 22.9514 | 23.5125 | 56.11 |
| Example 15 | 800 | 23.0020 | 23.5538 | 55.18 |
| Comparative | 800 | 23.0743 | 23.4496 | 37.53 |
| example 15 | | | | |
| Comparative | 800 | 22.9857 | 23.3602 | 37.45 |
| example 16 | | | | |
| Comparative | 800 | 23.1008 | 23.4670 | 36.62 |
| example 17 | | | | |

According to the evaluation results shown in Table 2, the drug delivery efficiencies of the intermittent drug delivery (Examples 7 to 15) were up to 50 to 75% and were significantly higher than that of the continuous drug delivery.

In the following evaluations, different levels of chronic obstructive pulmonary disease (COPD), including severe COPD, mild COPD, and general COPD, and interstitial lung disease (ILD) were simulated. The total amount of water was 1 mL, the volume of the air was 500 mL, other settings of the breathing simulator (I/E ratio and resistance), and the test results were shown in Table 3.

TABLE 3

| | | | | Weight of the | Weight of the | |
|---|---|---|---|---|---|---|
| | | | | filter paper | filter paper | |
| | | | | before | after | |
| | | | | outputting | outputting | |
| | | | | the nebulized | the nebulized | Drug delivery |
| | Mode | I/E ratio | Resistance | water (g) | water (g) | efficiency (%) |
| Example 16 | Severe | 1/4 | 20 | 23.0582 | 23.6758 | 61.76 |
| | COPD | | | | | |
| Example 17 | Severe | 1/4 | 20 | 24.7918 | 25.4053 | 61.35 |
| | COPD | | | | | |
| Example 18 | Severe | 1/4 | 20 | 25.1030 | 25.6989 | 59.59 |
| | COPD | | | | | |
| Comparative | Severe | 1/4 | 20 | 23.0883 | 23.3786 | 29.03 |
| example 18 | COPD | | | | | |
| Comparative | Severe | 1/4 | 20 | 23.0150 | 23.3042 | 28.92 |
| example 19 | COPD | | | | | |
| Comparative | Severe | 1/4 | 20 | 22.9956 | 23.2383 | 24.27 |
| example 20 | COPD | | | | | |
| Example 19 | mild | 1/4 | 5 | 25.0319 | 25.6338 | 60.19 |
| | COPD | | | | | |
| Example 20 | mild | 1/4 | 5 | 25.0650 | 25.6483 | 58.33 |
| | COPD | | | | | |
| Example 21 | mild | 1/4 | 5 | 24.9625 | 25.5857 | 62.32 |
| | COPD | | | | | |
| Comparative | mild | 1/4 | 5 | 24.6715 | 24.9548 | 28.33 |
| example 21 | COPD | | | | | |
| Comparative | mild | 1/4 | 5 | 25.0663 | 25.3721 | 30.58 |
| example 22 | COPD | | | | | |
| Comparative | mild | 1/4 | 5 | 25.1410 | 25.4354 | 29.44 |
| example 23 | COPD | | | | | |
| Example 22 | general | 1/5 | 20 | 25.2382 | 25.8492 | 61.1 |
| | COPD | | | | | |
| Comparative | general | 1/5 | 20 | 25.0867 | 25.3256 | 23.89 |
| example 24 | COPD | | | | | |
| Comparative | general | 1/5 | 20 | 24.8430 | 25.0993 | 25.63 |
| example 25 | COPD | | | | | |
| Comparative | general | 1/5 | 20 | 25.0807 | 25.3254 | 24.47 |
| example 26 | COPD | | | | | |
| Example 23 | ILD | 1/4 | 20 | 24.6750 | 25.3566 | 68.16 |
| Example 24 | ILD | 1/4 | 20 | 25.0173 | 25.6524 | 63.51 |
| Example 25 | ILD | 1/4 | 20 | 25.1464 | 25.8065 | 66.01 |
| Comparative | ILD | 1/4 | 20 | 25.1450 | 25.4767 | 33.17 |
| example 27 | | | | | | |
| Comparative | ILD | 1/4 | 20 | 24.9050 | 25.2134 | 30.84 |
| example 28 | | | | | | |
| Comparative | ILD | 1/4 | 20 | 24.7795 | 25.1013 | 32.18 |
| example 29 | | | | | | |

According to the evaluation results shown in Table 3, when the breathing simulator simulates different levels of COPD and ILD, the drug delivery efficiency of the intermittent drug delivery mode can still be significantly increased by 2 to 3 times. Therefore, the respiratory drug delivery device provided by the present invention can increase the utilization rate of the nebulized drugs entering the patient's respiratory system. The problem of the drug waste caused by continuous administration may be significantly improved. Moreover, the respiratory drug delivery device provided by the present invention utilizes wind shear sound generated by the patient's breathes as the standard for determining the patient's breathing condition, and the sound sensor is disposed on the inner surface or the outer surface of the air pipe; therefore, the sound sensor may not be affected by the noise produced by the patient's heartbeat of other appliances in the ward and can accurately determine the breathing frequency of the patient and deliver the drug intermittently.

What is claimed is:

1. A respiratory drug delivery device for delivering a drug to a patient's respiratory tract, comprising:
an inhalation part;
an air pipe, being connected to the inhalation part;
a sound sensor, being disposed on an inner surface or an outer surface of the air pipe for detecting a wind shear from the patient's breath and generating a sound signal; and
a drug delivery module, being connected to the air pipe and the sound sensor, wherein the drug delivery module nebulize the drug into a nebulized drug and output the nebulized drug to the air pipe;
wherein the drug delivery module output the nebulized drug at least when the patient inhales according to the sound signal.

2. The respiratory drug delivery device of claim 1, wherein the sound signal includes a plurality of breathing cycles, each of the breathing cycles sequentially includes an inhalation section, a pause section, and an exhalation section; wherein the drug delivery module outputs the nebulized drug at least at the inhalation section.

3. The respiratory drug delivery device of claim 2, wherein the drug delivery module outputs the nebulized drug at 0.1 to 5 seconds before a beginning of the inhalation section until an end of the inhalation section.

4. The respiratory drug delivery device of claim 3, wherein the drug delivery module outputs the nebulized drug at 0.1 to 5 seconds before the beginning of the inhalation section until 0.5 to 3 seconds before the end of the inhalation section.

5. The respiratory drug delivery device of claim 1, wherein the nebulized drug is at least one selected from a group consisting of a nebulized micronized drug, a nebulized water-soluble drug, and a nebulized drug suspension.

6. The respiratory drug delivery device of claim 5, wherein the drug delivery module includes a nebulizer for generating the nebulized drug.

7. The respiratory drug delivery device of claim 5, wherein a nozzle diameter of the nebulizer is below 10 μm.

8. The respiratory drug delivery device of claim 6, wherein the nebulizer is an ultrasonic vibrating atomizer.

9. The respiratory drug delivery device of claim 1, wherein the inhalation part is a nasal mask, an oronasal mask, or a nasal catheter.

10. A method for automating drug delivery to a respiratory system, comprising:
step (1): providing a respiratory drug delivery device, including an inhalation part; an air pipe being connected to the inhalation part; a sound sensor being disposed on an inner surface or an outer surface of the air pipe; and a drug delivery module outputting a nebulized drug to the air pipe;
step (2): collecting a respiratory flow with the air pipe through the inhalation part, detecting a wind shear from the patient's breath and generating a sound signal using the sound sensor; and
step (3): determining a breathing cycle of the patient from the sound signal; outputting the nebulized drug to the air pipe at least when the patient inhales for delivering the nebulized to the patient's respiratory system via the inhalation part.

11. The method of claim 10, wherein step (2), a respiratory frequency of the patient is 0.2 to 1 Hz.

12. The method of claim 11, wherein step (2), the respiratory flow is generated from the patient's nose and delivered to the air pipe.

13. The method of claim 10, wherein step (3), the sound signal includes a plurality of breathing cycles, each of the breathing cycles sequentially includes an inhalation section, a pause section, and an exhalation section, wherein the drug delivery module outputs the nebulized drug at least at the inhalation section.

14. The method of claim 13, wherein step (3), the drug delivery module outputs the nebulized drug at 0.1 to 5 seconds before a beginning of the inhalation section until an end of the inhalation section.

15. The method of claim 13, wherein step (3), the drug delivery module outputs the nebulized drug at 0.1 to 5 seconds before a beginning of the inhalation section until 0.5 to 3 seconds before an end of the inhalation section.
